SEQUENCE LISTING (2 sequences)

SEQ ID NO: 1 - protein, 1272 aa, Artificial Sequence
  recombinant botulinum neurotoxin subtype E with reduced persistence with MDM2 binding motif
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSLTFEHNWAQLENKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 2 - protein, 1272 aa, Artificial Sequence
  botulinum neurotoxin subtype E with reduced persistence
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSLTFEHNWAQLTSKSLVPRGSKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

(19) United States
(12) Patent Application Publication
(10) Pub. No.: US 2017/0112907 A1
(43) Pub. Date: Apr. 27, 2017

(54) NOVEL USES OF RECOMBINANT CLOSTRIDIAL NEUROTOXINS WITH DECREASED DURATION OF EFFECT

(71) Applicant: MERZ PHARMA GMBH & CO. KGAA, FRANKFURT AM MAIN (DE)

(72) Inventors: KLAUS FINK, KOELN (DE); SUSANNE GRAFE, DUESSELDORF (DE); IMKE SCHULTZ, RODGAU (DE)

(21) Appl. No.: 15/317,261

(22) PCT Filed: Jun. 12, 2015
(86) PCT No.: PCT/EP2015/001191

§ 371 Date: Dec. 8, 2016

(30) Foreign Application Priority Data
Jun. 13, 2014 (EP) 14002054.6

(51) Int. Cl. A61K 38/48; A61K 9/00

(52) U.S. Cl. CPC A61K 38/4893; A61K 9/0019

(57) ABSTRACT

This invention relates to novel uses of recombinant clostridial neurotoxins exhibiting decreased duration of effect, in particular uses for the treatment of different forms of back pain, in particular recurrent low back pain.

### FIELD OF THE INVENTION

This invention relates to novel uses of recombinant clostridial neurotoxins exhibiting decreased duration of effect, in particular uses for the treatment of different forms of back pain, in particular recurrent low back pain.

### BACKGROUND OF THE INVENTION

Clostridium is a genus of anaerobe gram-positive bacteria, belonging to the Firmicutes. Clostridium consists of around 100 species that include common free-living bacteria as well as important pathogens, such as Clostridium botulinum and Clostridium tetani. Both species produce neurotoxins, botulinum toxin and tetanus toxin, respectively. These neurotoxins are potent inhibitors of calcium-dependent neurotransmitter secretion of neuronal cells and are among the strongest toxins known to man. The lethal dose in humans lies between 0.1 ng and 1 ng per kilogram of body weight.

Oral ingestion of botulinum toxin via contaminated food or generation of botulinum toxin in wounds can cause botulism, which is characterised by paralysis of various muscles. Paralysis of the breathing muscles can cause death of the affected individual.

Although both botulinum neurotoxin (BoNT) and tetanus neurotoxin (TeNT) function via a similar initial physiological mechanism of action, inhibiting neurotransmitter release from the axon of the affected neuron into the synapse, they differ in their clinical response. While the botulinum toxin acts at the neuromuscular junction and other cholinergic synapses in the peripheral nervous system, inhibiting the release of the neurotransmitter acetylcholine and thereby causing flaccid paralysis, the tetanus toxin, which is transcytosed into central neurons, acts mainly in the central nervous system, preventing the release of the inhibitory neurotransmitters GABA (gamma-aminobutyric acid) and glycine by degrading the protein synaptobrevin. The consequent overactivity of spinal cord motor neurons causes generalized contractions of the agonist and antagonist musculature, termed a tetanic spasm (rigid paralysis).

While the tetanus neurotoxin exists in one immunologically distinct type, the botulinum neurotoxins are known to occur in seven different immunogenic serotypes, termed BoNT/A through BoNT/H with further subtypes. Most Clostridium botulinum strains produce one type of neurotoxin, but strains producing multiple toxins have also been described.

Botulinum and tetanus neurotoxins have highly homologous amino acid sequences and show a similar domain structure. Their biologically active form comprises two peptide chains, a light chain of about 50 kDa and a heavy chain of about 100 kDa, linked by a disulfide bond. A linker or loop region, whose length varies among different clostridial toxins, is located between the two cysteine residues forming the disulfide bond. This loop region is proteolytically cleaved by an unknown clostridial endoprotease to obtain the biologically active toxin.

The molecular mechanism of intoxication by TeNT and BoNT appears to be similar as well: entry into the target neuron is mediated by binding of the C-terminal part of the heavy chain to a specific cell surface receptor; the toxin is then taken up by receptor-mediated endocytosis. The low pH in the so formed endosome then triggers a conformational change in the clostridial toxin which allows it to embed itself in the endosomal membrane and to translocate through the endosomal membrane into the cytoplasm, where the disulfide bond joining the heavy and the light chain is reduced. The light chain can then selectively cleave so called SNARE-proteins, which are essential for different steps of neurotransmitter release into the synaptic cleft, e.g. recognition, docking and fusion of neurotransmitter-containing vesicles with the plasma membrane. TeNT, BoNT/B, BoNT/D, BoNT/F, and BoNT/G cause proteolytic cleavage of synaptobrevin or VAMP (vesicle-associated membrane protein), BoNT/A and BoNT/E cleave the plasma membrane-associated protein SNAP-25, and BoNT/C cleaves the integral plasma membrane protein syntaxin and SNAP-25.

In Clostridium botulinum, the botulinum toxin is formed as a protein complex comprising the neurotoxic component and non-toxic proteins. The accessory proteins embed the neurotoxic component thereby protecting it from degradation by digestive enzymes in the gastrointestinal tract. Thus, botulinum neurotoxins of most serotypes are orally toxic. Complexes with, for example, 450 kDa or with 900 kDa are obtainable from cultures of Clostridium botulinum.

In recent years, botulinum neurotoxins have been used as therapeutic agents, for example in the treatment of dystonias and spasms, and have additionally been used in cosmetic applications, such as the treatment of fine wrinkles. Preparations comprising botulinum toxin complexes are commercially available, e.g. from Ipsen Ltd (Dysport®) or Allergan Inc. (Botox®). A high purity neurotoxic component, free of any complexing proteins, is for example available from Merz Pharmaceuticals GmbH, Frankfurt (Xeomin®).

Clostridial neurotoxins are usually injected into the affected muscle tissue, bringing the agent close to the neuromuscular end plate, i.e. close to the cellular receptor mediating its uptake into the nerve cell controlling said affected muscle. Various degrees of neurotoxin spread have been observed. The neurotoxin spread is thought to depend on the injected amount and the particular neurotoxin preparation. It can result in adverse side effects such as paralysis in nearby muscle tissue, which can largely be avoided by reducing the injected doses to the therapeutically relevant level. Overdosing can also trigger the immune system to generate neutralizing antibodies that inactivate the neurotoxin preventing it from relieving the involuntary muscle activity. Immunologic tolerance to botulinum toxin has been shown to correlate with cumulative doses.

Clostridial neurotoxins display variable durations of action that are serotype specific. The clinical therapeutic effect of BoNT/A lasts approximately 3 months for neuromuscular disorders and 6 to 12 months for hyperhidrosis. The effects of BoNT/E, on the other hand, last about 4 weeks. One possible explanation for the divergent durations of action might be the distinct subcellular localizations of BoNT serotypes. The protease domain of BoNT/A light chain localizes in a punctate manner to the plasma membrane of neuronal cells, co-localizing with its substrate SNAP-25. In contrast, the short-duration BoNT/E serotype is cytoplasmic. Membrane association might protect BoNT/A from cytosolic degradation mechanisms allowing for prolonged persistence of BoNT/A in the neuronal cell.

The longer lasting therapeutic effect of BoNT/A makes it preferable for certain clinical uses and in particular for certain cosmetic uses compared to the other serotypes, for example serotypes B, C₁, D, E, F, G and H. On the other hand, it might be advantageous in certain scenarios to decrease the duration of the therapeutic effect of a botulinum neurotoxin in order to reduce the duration of muscle paralysis.

WO 2011/000929 and WO 2013/068476 describe neurotoxins exhibiting a shortened biological activity. In brief, the applications describe polypeptides comprising at least one E3 ligase recognition motif in the light chain, wherein said E3 ligase recognition motif is preferably a binding motif for the E3 ligase MDM2. Section [0006] of WO 2013/068476 generically lists a number of indications, which could potentially benefit from the application of modified neurotoxins with decreased duration of effect.

In particular, WO 2013/068476 describes variants of BoNT/E (SEQ ID NOs: 52 and 80 in WO 2013/068476), which were shown to have a duration of effect, which was decreased by about 25% compared to wild-type BoNT/E in a cell culture assay.

Despite the progress that has been made in the past in the treatment of indications that benefit from the intermittent paralysis of muscles, there is still a strong demand to further improve the therapeutic options available to the practitioner in the art, in particular in light of the fact that it might be desirable in certain indications, after an initial requirement for paralysing one or more muscles in such indication, to achieve an earlier recovery of muscle activity to assist the patient being treated in getting back to his or her normal life. To date, such aspects have not been addressed satisfactorily.

### OBJECTS OF THE INVENTION

It was an object of the invention to provide novel uses for recombinant clostridial neurotoxins exhibiting a decreased duration of effect, and to improve the treatment of different forms of back pain, in particular recurrent low back pain.

### SUMMARY OF THE INVENTION

The naturally occurring botulinum toxin serotypes display highly divergent durations of effect, with BoNT/A exhibiting the longest persistence, and BoNT/E exhibiting a comparatively short persistence. In order to broaden the applicability of botulinum neurotoxins, variants of BoNT/E have been created that exhibit a shorter duration of effect (see in particular WO 2013/068476).

Surprisingly, it has been identified that the variants disclosed in WO 2013/068476 might advantageously be used in particular situations, for which no satisfactory solution has been available so far.

Thus, the present invention relates to a botulinum neurotoxin subtype E with reduced persistence having a sequence according to SEQ ID NO: 1 or SEQ ID NO: 2, or a functionally active variant thereof, for use in the treatment of a patient, wherein the patient is suffering from back pain.

In a second aspect. the present invention relates to a method for the treatment of a patient suffering from back pain, comprising the step of administering a botulinum neurotoxin subtype E with reduced persistence having a sequence according to SEQ ID NO: 1 or SEQ ID NO: 2, or a functionally active variant thereof to said patient.

### DETAILED DESCRIPTION OF THE INVENTION

The present invention may be understood more readily by reference to the following detailed description of the invention and the examples included therein.

Thus, the present invention relates to a botulinum neurotoxin subtype E with reduced persistence having a sequence according to SEQ ID NO: 1 or SEQ ID NO: 2, or a functionally active variant thereof, for use in the treatment of a patient, wherein the patient is suffering from back pain.

In a second aspect. the present invention relates to a method for the treatment of a patient suffering from back pain, comprising the step of administering a botulinum neurotoxin subtype E with reduced persistence having a sequence according to SEQ ID NO: 1 or SEQ ID NO: 2, or a functionally active variant thereof to said patient.

In the context of the present invention, the term “functionally active variant” refers to a neurotoxin, in particular a recombinant neurotoxin, that differs in the amino acid sequence and/or the nucleic acid sequence encoding the amino acid sequence from the botulinum neurotoxin subtype E with reduced persistence according to SEQ ID NO: 1 or SEQ ID NO: 2, but is still functionally active. In the context of the present invention, the term “functionally active” refers to the property of such recombinant clostridial neurotoxin variant to (i) achieve muscle paralysis to at least 50%, particularly to at least 60%, at least 70%, at least 80%, and most particularly at least 90% of the muscle paralysis achieved with the same amount of a botulinum neurotoxin subtype E with reduced persistence according to SEQ ID NO: 1 or SEQ ID NO: 2, and (ii) achieve such muscle paralysis for a duration of time that is at maximum 10% shorter or longer, particularly at maximum 5% shorter or longer than the duration of paralysis achieved by a botulinum neurotoxin subtype E with reduced persistence according to SEQ ID NO: 1 or SEQ ID NO: 2 (i.e. which shows between 90% and 110% of the duration of paralysis, particularly between 95% and 105% of the duration of paralysis achieved by a botulinum neurotoxin subtype E with reduced persistence according to SEQ ID NO: 1 or SEQ ID NO: 2).

On the protein level, a functionally active variant will maintain key features of the corresponding parental clostridial neurotoxin, such as key residues for the endopeptidase activity in the light chain, or key residues for the attachment to the neurotoxin receptors or for translocation through the endosomal membrane in the heavy chain, but may contain one or more mutations comprising a deletion of one or more amino acids of the corresponding clostridial neurotoxin, an addition of one or more amino acids of the corresponding clostridial neurotoxin, and/or a substitution of one or more amino acids of the corresponding clostridial neurotoxin. Particularly, said deleted, added and/or substituted amino acids are consecutive amino acids. According to the teaching of the present invention, any number of amino acids may be added, deleted, and/or substituted, as long as the functionally active variant remains biologically active as defined above. For example, 1, 2, 3, 4, 5, up to 10, up to 15, up to 25, up to 50, up to 100, up to 200, up to 400, up to 500 amino acids or even more amino acids of a botulinum neurotoxin subtype E with reduced persistence according to SEQ ID NO: 1 or SEQ ID NO: 2 may be added, deleted, and/or substituted. This neurotoxin fragment may contain an N-terminal, C-terminal, and/or one or more internal deletion(s).

In another embodiment, the functional variant of a clostridial neurotoxin additionally comprises a signal peptide. Usually, said signal peptide will be located at the N-terminus of the neurotoxin. Many such signal peptides are known in the art and are comprised by the present invention. In particular, the signal peptide results in transport of the neurotoxin across a biological membrane, such as the membrane of the endoplasmic reticulum, the Golgi membrane or the plasma membrane of a eukaryotic or prokaryotic cell. It has been found that signal peptides, when attached to the neurotoxin, will mediate secretion of the neurotoxin into the supernatant of the cells. In certain embodiments, the signal peptide will be cleaved off in the course of, or subsequent to, secretion, so that the secreted protein lacks the N-terminal signal peptide, is composed of separate light and heavy chains, which are covalently linked by disulfide bridges, and is proteolytically active.

In particular embodiments, the functional variant has in its clostridium neurotoxin part a sequence identity of at least 40%, at least 50%, at least 60%, at least 70% or most particularly at least 80%, and a sequence homology of at least 60%, at least 70%, at least 80%, at least 90%, or most particularly at least 95% to the corresponding part of a botulinum neurotoxin subtype E with reduced persistence according to SEQ ID NO: 1 or SEQ ID NO: 2. Methods and algorithms for determining sequence identity and/or homology, including the comparison of variants having deletions, additions, and/or substitutions relative to a parental sequence, are well known to the practitioner of ordinary skill in the art. On the DNA level, the nucleic acid sequences encoding the functional homologue and the parental clostridial neurotoxin may differ to a larger extent due to the degeneracy of the genetic code. It is known that the usage of codons is different between prokaryotic and eukaryotic organisms. Thus, when expressing a prokaryotic protein such as a clostridial neurotoxin, in a eukaryotic expression system, it may be necessary, or at least helpful, to adapt the nucleic acid sequence to the codon usage of the expression host cell, meaning that sequence identity or homology may be rather low on the nucleic acid level.

In the context of the present invention, the term “variant” refers to a neurotoxin that is a chemically, enzymatically, or genetically modified derivative of a botulinum neurotoxin subtype E with reduced persistence according to SEQ ID NO: 1 or SEQ ID NO: 2. A chemically modified derivative may be one that is modified by pyruvation, phosphorylation, sulfatation, lipidation, pegylation, glycosylation and/or the chemical addition of an amino acid or a polypeptide comprising between 2 and 100 amino acids, including modification occurring in the eukaryotic host cell used for expressing the derivative. An enzymatically modified derivative is one that is modified by the activity of enzymes, such as endo- or exoproteolytic enzymes, including modification by enzymes of the eukaryotic host cell used for expressing the derivative. As pointed out above, a genetically modified derivative is one that has been modified by deletion or substitution of one or more amino acids contained in, or by addition of one or more amino acids (including polypeptides comprising between 2 and about 100 amino acids) to, the amino acid sequence of said clostridial neurotoxin. Methods for designing and constructing such chemically or genetically modified derivatives and for testing of such variants for functionality are well known to anyone of ordinary skill in the art.

In the context of the present invention, the term “recombinant neurotoxin” refers to a composition comprising a clostridial neurotoxin that is obtained by expression of the neurotoxin in a heterologous cell such as E. coli, and including, but not limited to, the raw material obtained from a fermentation process (supernatant, composition after cell lysis), a fraction comprising a clostridial neurotoxin obtained from separating the ingredients of such a raw material in a purification process, an isolated and essentially pure protein, and a formulation for pharmaceutical and/or aesthetic use comprising a clostridial neurotoxin and additionally pharmaceutically acceptable solvents and/or excipients.

In the context of the present invention, the term “comprises” or “comprising” means “including, but not limited to”. The term is intended to be open-ended, to specify the presence of any stated features, elements, integers, steps or components, but not to preclude the presence or addition of one or more other features, elements, integers, steps, components, or groups thereof. The term “comprising” thus includes the more restrictive terms “consisting of” and “consisting essentially of”.

In the context of the present invention, the term “botulinum neurotoxin subtype E” refers to a particular neurotoxin found in and obtainable from Clostridium botulinum having a sequence shown in SEQ ID NO: 82 of WO 2013/068476.

In particular embodiments, said functionally active variant has a persistence that is at maximum 5% shorter or longer than the duration of paralysis achieved by a botulinum neurotoxin subtype E with reduced persistence according to SEQ ID NO: 1 or SEQ ID NO: 2.

Without wishing to be bound by theory, the recombinant clostridial neurotoxins of the present invention might show decreased biological half-life, increased degradation rates, increased diffusion rates, decreased uptake by neuronal cells, and/or modified intracellular translocation rates, in each case relative to wild-type botulinum neurotoxin of subtype E (BoNT/E).

Back pain is neither a disease nor a diagnostic entity in itself, but rather a constellation of symptoms. The pain may originate from bones, facet joints, nerves, muscles, ligaments, blood vessels or other components of the back.

Back pain can be categorized according to the location, the duration of pain, the etiology of the disease, and the mechanism of pain.

Back pain is classically stratified into upper and low back pain and then by duration (acute, subacute or chronic). In the context of the present invention, the term “back pain” further includes neck pain and pain caused by scoliosis.

Upper back pain occurs between the bottom of the neck and top of the lumbar spine, whereas low back pain occurs below the lowest rib and above the inferior gluteal folds

Neck pain is a pain sensation felt in the neck, which may originate from a range of different causes, including spinal problems, joint disruptions or tightness of the muscles in the neck as well as in the upper back, or pinching of nerves.

Scoliosis is a medical condition in which the axis of the spine of a patient suffering from that condition has a three-dimensional deviation, which may result in acute or chronic pain sensations.

With respect to the etiology of back pain, one can differentiate (i) specific back pain, which is caused by a specific pathophysiological mechanism (e.g., systemic disease, infection, injury, trauma or structural deformity), and (ii) non-specific or mechanical back pain, which is due to an unknown cause and/or is diagnosed based on exclusion of specific pathology, and which includes pain related to disc degeneration or herniation.

With respect to the duration of back pain, one can differentiate (i) acute pain, which is characterized by a sudden onset, which often resolves in less than 6 weeks, and which is often self-limiting, (ii) subacute pain, which lasts between 6 and 12 weeks; and (iii) chronic pain, which persists for more than 12 weeks, and which often is the result of degenerative or traumatic conditions of the spine.

With respect to the mechanism of back pain, one can differentiate (i) nociceptive pain, which is caused by injury (e.g. by a cut, bruise, fracture, or burn); (ii) neuropathic pain, which is pain initiated or caused by a primary lesion or dysfunction in the nervous system; (iii) psychogenic pain, which is caused by a psychological process, which, however, is rare and generally only in persons with a mental disorder.

Thus, in particular embodiments, said patient is suffering from low back pain, upper back pain, neck pain or pain caused by scoliosis, particularly from low back pain.

In particular embodiments, said patient is suffering from low back pain, wherein said patient experiences low back pain recurring within a period of from 6 weeks to 12 weeks after a first occurrence of low back pain.

Recurring Acute Non-specific Low Back Pain is a particular form of subacute and non-specific low back pain that is recurrent, i.e., a non-specific low back pain (NSLBP) patient that resolves and then comes back with similar acute NSLBP on a repeated basis. Subacute/recurring NSLBP represents the 10% of back pain that is not adequately treated with first line options such as (a) oral anti-inflammatory NSAIDs or acetaminophen (APAP), NSARs; (b) mild short-acting opioids (e.g., Tramadol/Ultram); (c) opioid-APAP combination products, and (d) systemically acting muscle relaxing drugs. Subacute/recurring acute NSLBP is typically referred to a specialist, generally an orthopedic surgeon, physical medicine/rehabilitation specialist or rheumatologist.

The second line treatment includes as standard of care aggressive physical therapy, and in addition glucocorticoid injections and muscle relaxants, however this therapy is often not tolerated. Physicians encourage their patients to resume normal activities as soon as possible. Back pain specialists are generally more aggressive with physical therapy and exercise regimens as soon as the pain has started to subside, but has not necessarily completely stopped. The goal of physical therapy/exercise is to strengthen the back muscles to prevent subsequent injury.

Efficacy of steroid injections is limited, so patients must receive repeat injections. However, steroid injections are usually limited to just three per year (at $150 per injection) because there is a chance that they may weaken spinal bones and nearby muscles. Steroid injections also suppress the body's natural hormone balance and can lead to adrenal insufficiency. Delaying repeat injections allows the patient's body to return to its normal balance. The risk of these side effects may increase with the number of steroid injections received and the dose given in each injection.

Thus, there are significant unmet needs for subacute/recurring acute NSLBP, as the pain has persisted longer than the typical case and/or the pain recurs at an unacceptable rate. In particular, the following issues frequently arise in the context of treating low back pain patients:
side effects of muscle relaxants and/or benzodiazepines;
limited efficacy: only symptomatic effect, no long-term effects, e.g. efficacy of steroid injections is limited, so patients must receive injections repeatedly; and/or
concerns over abuse potential.

The proteins according to SEQ ID NO: 1 or SEQ ID NO: 2 are local muscle relaxants with an onset of effect within a day and an estimated duration of effect of 4 weeks (+2 weeks). An injection of one of these proteins relieves muscle spasm leading to an improvement of pain which allows physical therapy earlier. The advantages of such treatment are:
better efficacy and tolerability compared to steroid injections:
   Steroid injections are usually limited to just three per year (at $150 per injection) because there is a chance that they may weaken spinal bones and nearby muscles.
   Steroid injections also suppress the body's natural hormone balance and can lead to adrenal insufficiency.
   Delaying repeat injections allows the patient's body to return to its normal balance.
   The risk of these side effects may increase with the number of steroid injections received and the dose given in each injection
   If the patient is in pain, but it is too early to safely receive the next steroid injection, the proteins according to SEQ ID NO: 1 or SEQ ID NO: 2 are good options.
better tolerability compared to muscle relaxants or opioids (e.g., confusion and dizziness with benzodiazepine muscle relaxants, euphoria with opioids).
no significant safety issues (lack of abuse potential, does not diminish functional recovery, lack of systemic side effects).

In particular embodiments, said treatment comprises the administration of said botulinum neurotoxin subtype E with reduced persistence according to SEQ ID NO: 1 or SEQ ID NO: 2, or of said functionally active variant thereof, to one or more muscles selected from the list of: trapezius, latissimus dorsi, rhomboideus, iliocostalis, longissimus, spinalis, semispinalis, multifidi, rotatores, intertransversarii, erector spinae and superficial flexors (sternocleidomastoideus, anterior scalene muscles).

In particular embodiments, muscle paralysis by a botulinum neurotoxin of more than 5 weeks, in particular of more than 4 weeks, and more particularly of more than 3 weeks, is contraindicated and/or deemed to be associated with negative impact on overall treatment success, particularly due to high likelihood of increased muscle atrophy.

### Examples

### Example 1: Treatment of Low Back Pain

A patient has low back pain and is treated with NSAR and steroids for more than 6 weeks without success. The physical examination reveals contracture in the paravertebral muscles with 4 trigger points. Within one day after injection at each trigger point of a botulinum neurotoxin subtype E with reduced persistence having a sequence according to SEQ ID NO: 1, the pain improves.

After two weeks he can start physiotherapy with further improvement of pain, and after four weeks, he is able to get back to work fully recovered.

### Example 2: Treatment of Upper Back Pain

A patient has upper back pain and is treated with NSAR and steroids for more than 6 weeks without success. The physical examination reveals contractures in M. rhomboideus minor and major identifying 8 trigger points. Within one day after injection at each trigger point of a botulinum neurotoxin subtype E with reduced persistence having a sequence according to SEQ ID NO: 1, the pain improves.

After two weeks he can start physiotherapy with further improvement of pain, and after four weeks, he is able to get back to work fully recovered.

### Example 3: Design of Clinical Trials

A phase 1 dose-response using an accepted model by the Regulatory Agencies study is foreseen because classical PK/PD phase 1 studies are not possible with botulinum neurotoxins. Dose-response profile and duration of effect as well as systemic diffusion in adjacent muscles after a single intramuscular injection of the protein according to SEQ ID NO: 1 in three to four concentrations into the Extensor Digitorum brevis (EDB) muscle will be investigated in healthy male volunteers in a single center, double-blind randomized study.

The planned observation period is up to 12 weeks after injection. Study parameters are the EDB-Compound Muscle Action Potential (CMAP) M-wave amplitude, Abductor hallucis-CMAP M-wave amplitude, Abductor digiti quinti-CMAP M-wave amplitude, and Adverse Events.

The investigation of safety, tolerability and efficacy of the protein according to SEQ ID NO: 1 in subacute low back pain repair is the aim of a Phase 2a study with a randomized, double-blind, placebo-controlled, parallel group design. Up to 60 patients will be injected with 200 U of the protein according to SEQ ID NO: 1. Pain Scales and QoL Assessments, and amount of physiotherapy will be investigated 2, 6, 12, 18, and 24 weeks after injection.

SEQUENCES

```
SEQ ID NO. 1
Met Pro Lys Ile Asn Ser Phe Asn Tyr Asn Asp Pro Val Asn Asp Arg
1 5 10 15
Thr Ile Leu Tyr Ile Lys Pro Gly Gly Cys Gln Glu Phe Tyr Lys Ser
                        20 25 30
Phe Asn Ile Met Lys Asn Ile Trp Ile Ile Pro Glu Arg Asn Val Ile
                35 40 45
Gly Thr Thr Pro Gln Asp Phe His Pro Pro Thr Ser Leu Lys Asn Gly
        50 55 60
Asp Ser Ser Tyr Tyr Asp Pro Asn Tyr Leu Gln Ser Asp Glu Glu Lys
65 70 75 80
Asp Arg Phe Leu Lys Ile Val Thr Lys Ile Phe Asn Arg Ile Asn Asn
                                85 90 95
Asn Leu Ser Gly Gly Ile Leu Leu Glu Glu Leu Ser Lys Ala Asn Pro
                        100 105 110
Tyr Leu Gly Asn Asp Asn Thr Pro Asp Asn Gln Phe His Ile Gly Asp
                115 120 125
Ala Ser Ala Val Glu Ile Lys Phe Ser Asn Gly Ser Gln Asp Ile Leu
        130 135 140
Leu Pro Asn Val Ile Ile Met Gly Ala Glu Pro Asp Leu Phe Glu Thr
145 150 155 160
Asn Ser ser Asn Ile Ser Leu Arg Asn Asn Tyr Met Pro ser Asn His
                                165 170 175
Gly Phe Gly Ser Ile Ala Ile Val Thr Phe Ser Pro Glu Tyr Ser Phe
                        180 185 190
Arg Phe Asn Asp Asn Ser Met Asn Glu Phe Ile Gln Asp Pro Ala Leu
                195 200 205
Thr Leu Met His Glu Leu Ile His Ser Leu His Gly Leu Tyr Gly Ala
        210 215 220
Lys Gly Ile Thr Thr Lys Tyr Thr Ile Thr Gln Lys Gln Asn Pro Leu
225 230 235 240
Ile Thr Asn Ile Arg Gly Thr Asn Ile Glu Glu Phe Leu Thr Phe Gly
                                245 250 255
Gly Thr Asp Leu Asn Ile Ile Thr Ser Ala Gln Ser Asn Asp Ile Tyr
                        260 265 270
Thr Asn Leu Leu Ala Asp Tyr Lys Lys Ile Ala Ser Lys Leu Ser Lys
                275 280 285
Val Gln Val Ser Asn Pro Leu Leu Asn Pro Tyr Lys Asp Val Phe Glu
        290 295 300
Ala Lys Tyr Gly Leu Asp Lys Asp Ala Ser Gly Ile Tyr Ser Val Asn
305 310 315 320
Ile Asn Lys Phe Asn Asp Ile Phe Lys Lys Leu Tyr ser Phe Thr Glu
                                325 330 335
Phe Asp Leu Ala Thr Lys Phe Gln Val Lys Cys Arg Gln Thr Tyr Ile
                        340 345 350
Gly Gln Tyr Lys Tyr Phe Lys Leu Ser Asn Leu Leu Asn Asp ser Ile
                355 360 365
Tyr Asn Ile Ser Glu Gly Tyr Asn Ile Asn Asn Leu Lys Val Asn Phe
        370 375 380
Arg Gly Gln Asn Ala Asn Leu Asn Pro Arg Ile Ile Thr Pro Ile Thr
385 390 395 400
Gly Arg Gly Leu Val Lys Lys Ile Ile Arg Phe Cys Val Arg Gly Ile
                                405 410 415
Ile Thr Ser Leu Thr Phe Glu His Asn Trp Ala Gln Leu Glu Asn Lys
                        420 425 430
Ser Leu Val Pro Arg Gly Ser Lys Ala Leu Asn Asp Leu Cys Ile Glu
                435 440 445
Ile Asn Asn Gly Glu Leu Phe Phe Val Ala Ser Glu Asn Ser Tyr Asn
        450 455 460
Asp Asp Asn Ile Asn Thr Pro Lys Glu Ile Asp Asp Thr Val Thr Ser
465 470 475 480
Asn Asn Asn Tyr Glu Asn Asp Leu Asp Gln Val Ile Leu Asn Phe Asn
                                485 490 495
Ser Glu Ser Ala Pro Gly Leu Ser Asp Glu Lys Leu Asn Leu Thr Ile
                        500 505 510
Gln Asn Asp Ala Tyr Ile Pro Lys Tyr Asp Ser Asn Gly Thr Ser Asp
                515 520 525
Ile Glu Gln His Asp Val Asn Glu Leu Asn Val Phe Phe Tyr Leu Asp
        530 535 540
Ala Gln Lys Val Pro Glu Gly Glu Asn Asn Val Asn Leu Thr Ser Ser
545 550 555 560
Ile Asp Thr Ala Leu Leu Glu Gln Pro Lys Ile Tyr Thr Phe Phe Ser
                                565 570 575
Ser Glu Phe Ile Asn Asn Val Asn Lys Pro Val Gln Ala Ala Leu Phe
                        580 585 590
Val Ser Trp Ile Gln Gln Val Leu Val Asp Phe Thr Thr Glu Ala Asn
                595 600 605
Gln Lys Ser Thr Val Asp Lys Ile Ala Asp Ile Ser Ile Val Val Pro
        610 615 620
Tyr Ile Gly Leu Ala Leu Asn Ile Gly Asn Glu Ala Gln Lys Gly Asn
625 630 635 640
Phe Lys Asp Ala Leu Glu Leu Leu Gly Ala Gly Ile Leu Leu Glu Phe
                                645 650 655
Glu Pro Glu Leu Leu Ile Pro Thr Ile Leu Val Phe Thr Ile Lys Ser
                        660 665 670
Phe Leu Gly Ser Ser Asp Asn Lys Asn Lys Val Ile Lys Ala Ile Asn
                675 680 685
Asn Ala Leu Lys Glu Arg Asp Glu Lys Trp Lys Glu Val Tyr Ser Phe
        690 695 700
Ile Val Ser Asn Trp Met Thr Lys Ile Asn Thr Gln Phe Asn Lys Arg
705 710 715 720
Lys Glu Gln Met Tyr Gln Ala Leu Gln Asn Gln Val Asn Ala Ile Lys
                                725 730 735
Thr Ile Ile Glu Ser Lys Tyr Asn Ser Tyr Thr Leu Glu Glu Lys Asn
                        740 745 750
Glu Leu Thr Asn Lys Tyr Asp Ile Lys Gln Ile Glu Asn Glu Leu Asn
                755 760 765
Gln Lys Val Ser Ile Ala Met Asn Asn Ile Asp Arg Phe Leu Thr Glu
        770 775 780
Ser Ser Ile Ser Tyr Leu Met Lys Leu Ile Asn Glu Val Lys Ile Asn
785 790 795 800
Lys Leu Arg Glu Tyr Asp Glu Asn Val Lys Thr Tyr Leu Leu Asn Tyr
                                805 810 815
Ile Ile Gln His Gly Ser Ile Leu Gly Glu Ser Gln Gln Glu Leu Asn
                        820 825 830
Ser Met Val Thr Asp Thr Leu Asn Asn Ser Ile Pro Phe Lys Leu Ser
                835 840 845
Ser Tyr Thr Asp Asp Lys Ile Leu Ile Ser Tyr Phe Asn Lys Phe Phe
        850 855 860
Lys Arg Ile Lys Ser Ser Ser Val Leu Asn Met Arg Tyr Lys Asn Asp
865 870 875 880
Lys Tyr Val Asp Thr Ser Gly Tyr Asp Ser Asn Ile Asn Ile Asn Gly
                                885 890 895
Asp Val Tyr Lys Tyr Pro Thr Asn Lys Asn Gln Phe Gly Ile Tyr Asn
                        900 905 910
Asp Lys Leu Ser Glu Val Asn Ile Ser Gln Asn Asp Tyr Ile Ile Tyr
                915 920 925
Asp Asn Lys Tyr Lys Asn Phe Ser Ile Ser Phe Trp Val Arg Ile Pro
        930 935 940
Asn Tyr Asp Asn Lys Ile Val Asn Val Asn Asn Glu Tyr Thr Ile Ile
945 950 955 960
Asn Cys Met Arg Asp Asn Asn Ser Gly Trp Lys Val Ser Leu Asn His
                                965 970 975
Asn Glu Ile Ile Trp Thr Leu Gln Asp Asn Ala Gly Ile Asn Gln Lys
                        980 985 990
Leu Ala Phe Asn Tyr Gly Asn Ala Asn Gly Ile Ser Asp Tyr Ile Asn
                995 1000 1005
Lys Trp Ile Phe Val Thr Ile Thr Asn Asp Arg Leu Gly Asp Ser
        1010 1015 1020
Lys Leu Tyr Ile Asn Gly Asn Leu Ile Asp Gln Lys Ser Ile Leu
        1025 1030 1035
Asn Leu Gly Asn Ile His Val Ser Asp Asn Ile Leu Phe Lys Ile
        1040 1045 1050
Val Asn Cys Ser Tyr Thr Arg Tyr Ile Gly Ile Arg Tyr Phe Asn
        1055 1060 1065
Ile Phe Asp Lys Glu Leu Asp Glu Thr Glu Ile Gln Thr Leu Tyr
        1070 1075 1080
Ser Asn Glu Pro Asn Thr Asn Ile Leu Lys Asp Phe Trp Gly Asn
        1085 1090 1095
Tyr Leu Leu Tyr Asp Lys Glu Tyr Tyr Leu Leu Asn Val Leu Lys
        1100 1105 1110
Pro Asn Asn Phe Ile Asp Arg Arg Lys Asp Ser Thr Leu Ser Ile
        1115 1120 1125
Asn Asn Ile Arg ser Thr Ile Leu Leu Ala Asn Arg Leu Tyr Ser
        1130 1135 1140
Gly Ile Lys val Lys Ile Gln Arg val Asn Asn Ser Ser Thr Asn
        1145 1150 1155
Asp Asn Leu Val Arg Lys Asn Asp Gln Val Tyr Ile Asn Phe val
        1160 1165 1170
Ala Ser Lys Thr His Leu Phe Pro Leu Tyr Ala Asp Thr Ala Thr
        1175 1180 1185
Thr Asn Lys Glu Lys Thr Ile Lys Ile Ser Ser Ser Gly Asn Arg
        1190 1195 1200
Phe Asn Gln Val Val Val Met Asn Ser Val Gly Asn Asn Cys Thr
        1205 1210 1215
Met Asn Phe Lys Asn Asn Asn Gly Asn Asn Ile Gly Leu Leu Gly
        1220 1225 1230
Phe Lys Ala Asp Thr Val Val Ala Ser Thr Trp Tyr Tyr Thr His
        1235 1240 1245
Met Arg Asp His Thr Asn Ser Asn Gly Cys Phe Trp Asn Phe Ile
        1250 1255 1260
Ser Glu Glu His Gly Trp Gln Glu Lys
        1265 1270
SEQ ID NO. 2
Met Pro Lys Ile Asn Ser Phe Asn Tyr Asn Asp Pro Val Asn Asp Arg
1 5 10 15
Thr Ile Leu Tyr Ile Lys Pro Gly Gly Cys Gln Glu Phe Tyr Lys Ser
                        20 25 30
Phe Asn Ile Met Lys Asn Ile Trp Ile Ile Pro Glu Arg Asn Val Ile
                35 40 45
Gly Thr Thr Pro Gln Asp Phe His Pro Pro Thr Ser Leu Lys Asn Gly
        50 55 60
Asp Ser Ser Tyr Tyr Asp Pro Asn Tyr Leu Gln Ser Asp Glu Glu Lys
65 70 75 80
Asp Arg Phe Leu Lys Ile Val Thr Lys Ile Phe Asn Arg Ile Asn Asn
                                85 90 95
Asn Leu Ser Gly Gly Ile Leu Leu Glu Glu Leu Ser Lys Ala Asn Pro
                        100 105 110
Tyr Leu Gly Asn Asp Asn Thr Pro Asp Asn Gln Phe His Ile Gly Asp
                115 120 125
Ala Ser Ala Val Glu Ile Lys Phe Ser Asn Gly Ser Gln Asp Ile Leu
        130 135 140
Leu Pro Asn val Ile Ile Met Gly Ala Glu Pro Asp Leu Phe Glu Thr
145 150 155 160
Asn Ser Ser Asn Ile Ser Leu Arg Asn Asn Tyr met Pro Ser Asn His
                                165 170 175
Gly Phe Gly Ser Ile Ala Ile Val Thr Phe Ser Pro Glu Tyr Ser Phe
                        180 185 190
Arg Phe Asn Asp Asn Ser Met Asn Glu Phe Ile Gln Asp Pro Ala Leu
                195 200 205
Thr Leu met His Glu Leu Ile His ser Leu His Gly Leu Tyr Gly Ala
        210 215 220
Lys Gly Ile Thr Thr Lys Tyr Thr Ile Thr Gln Lys Gln Asn Pro Leu
225 230 235 240
Ile Thr Asn Ile Arg Gly Thr Asn Ile Glu Glu Phe Leu Thr Phe Gly
                                245 250 255
Gly Thr Asp Leu Asn Ile Ile Thr Ser Ala Gln Ser Asn Asp Ile Tyr
                        260 265 270
Thr Asn Leu Leu Ala Asp Tyr Lys Lys Ile Ala Ser Lys Leu Ser Lys
                275 280 285
Val Gln Val Ser Asn Pro Leu Leu Asn Pro Tyr Lys Asp Val Phe Glu
        290 295 300
Ala Lys Tyr Gly Leu Asp Lys Asp Ala ser Gly Ile Tyr Ser Val Asn
305 310 315 320
Ile Asn Lys Phe Asn Asp Ile Phe Lys Lys Leu Tyr Ser Phe Thr Glu
                                325 330 335
Phe Asp Leu Ala Thr Lys Phe Gln Val Lys Cys Arg Gln Thr Tyr Ile
                        340 345 350
Gly Gln Tyr Lys Tyr Phe Lys Leu Ser Asn Leu Leu Asn Asp Ser Ile
                355 360 365
Tyr Asn Ile Ser Glu Gly Tyr Asn Ile Asn Asn Leu Lys Val Asn Phe
        370 375 380
Arg Gly Gln Asn Ala Asn Leu Asn Pro Arg Ile Ile Thr Pro Ile Thr
385 390 395 400
Gly Arg Gly Leu Val Lys Lys Ile Ile Arg Phe Cys val Arg Gly Ile
                                405 410 415
Ile Thr Ser Leu Thr Phe Glu His Asn Trp Ala Gln Leu Thr Ser Lys
                        420 425 430
Ser Leu Val Pro Arg Gly ser Lys Ala Leu Asn Asp Leu Cys Ile Glu
                435 440 445
Ile Asn Asn Gly Glu Leu Phe Phe val Ala Ser Glu Asn ser Tyr Asn
        450 455 460
Asp Asp Asn Ile Asn Thr Pro Lys Glu Ile Asp Asp Thr Val Thr Ser
465 470 475 480
Asn Asn Asn Tyr Glu Asn Asp Leu Asp Gln Val Ile Leu Asn Phe Asn
                                485 490 495
Ser Glu ser Ala Pro Gly Leu Ser Asp Glu Lys Leu Asn Leu Thr Ile
                        500 505 510
Gln Asn Asp Ala Tyr Ile Pro Lys Tyr Asp Ser Asn Gly Thr Ser Asp
                515 520 525
Ile Glu Gln His Asp Val Asn Glu Leu Asn Val Phe Phe Tyr Leu Asp
        530 535 540
Ala Gln Lys Val Pro Glu Gly Glu Asn Asn Val Asn Leu Thr Ser Ser
545 550 555 560
Ile Asp Thr Ala Leu Leu Glu Gln Pro Lys Ile Tyr Thr Phe Phe Ser
                                565 570 575
Ser Glu Phe Ile Asn Asn Val Asn Lys Pro Val Gln Ala Ala Leu Phe
                        580 585 590
Val Ser Trp Ile Gln Gln Val Leu Val Asp Phe Thr Thr Glu Ala Asn
                595 600 605
Gln Lys Ser Thr Val Asp Lys Ile Ala Asp Ile Ser Ile Val Val Pro
        610 615 620
Tyr Ile Gly Leu Ala Leu Asn Ile Gly Asn Glu Ala Gln Lys Gly Asn
625 630 635 640
Phe Lys Asp Ala Leu Glu Leu Leu Gly Ala Gly Ile Leu Leu Glu Phe
                                645 650 655
Glu Pro Glu Leu Leu Ile Pro Thr Ile Leu Val Phe Thr Ile Lys Ser
                        660 665 670
Phe Leu Gly Ser Ser Asp Asn Lys Asn Lys Val Ile Lys Ala Ile Asn
                675 680 685
Asn Ala Leu Lys Glu Arg Asp Glu Lys Trp Lys Glu Val Tyr Ser Phe
        690 695 700
Ile Val Ser Asn Trp Met Thr Lys Ile Asn Thr Gln Phe Asn Lys Arg
705 710 715 720
Lys Glu Gln Met Tyr Gln Ala Leu Gln Asn Gln Val Asn Ala Ile Lys
                                725 730 735
Thr Ile Ile Glu Ser Lys Tyr Asn Ser Tyr Thr Leu Glu Glu Lys Asn
                        740 745 750
Glu Leu Thr Asn Lys Tyr Asp Ile Lys Gln Ile Glu Asn Glu Leu Asn
                755 760 765
Gln Lys Val Ser Ile Ala Met Asn Asn Ile Asp Arg Phe Leu Thr Glu
        770 775 780
Ser Ser Ile Ser Tyr Leu Met Lys Leu Ile Asn Glu Val Lys Ile Asn
785 790 795 800
Lys Leu Arg Glu Tyr Asp Glu Asn Val Lys Thr Tyr Leu Leu Asn Tyr
                                805 810 815
Ile Ile Gln His Gly Ser Ile Leu Gly Glu Ser Gln Gln Glu Leu Asn
                        820 825 830
Ser Met Val Thr Asp Thr Leu Asn Asn Ser Ile Pro Phe Lys Leu Ser
                835 840 845
Ser Tyr Thr Asp Asp Lys Ile Leu Ile Ser Tyr Phe Asn Lys Phe Phe
        850 855 860
Lys Arg Ile Lys Ser Ser Ser Val Leu Asn Met Arg Tyr Lys Asn Asp
865 870 875 880
Lys Tyr Val Asp Thr Ser Gly Tyr Asp Ser Asn Ile Asn Ile Asn Gly
                                885 890 895
Asp Val Tyr Lys Tyr Pro Thr Asn Lys Asn Gln Phe Gly Ile Tyr Asn
                        900 905 910
Asp Lys Leu Ser Glu Val Asn Ile Ser Gln Asn Asp Tyr Ile Ile Tyr
                915 920 925
Asp Asn Lys Tyr Lys Asn Phe Ser Ile Ser Phe Trp Val Arg Ile Pro
        930 935 940
Asn Tyr Asp Asn Lys Ile Val Asn Val Asn Asn Glu Tyr Thr Ile Ile
945 950 955 960
Asn Cys Met Arg Asp Asn Asn Ser Gly Trp Lys Val Ser Leu Asn His
                                965 970 975
Asn Glu Ile Ile Trp Thr Leu Gln Asp Asn Ala Gly Ile Asn Gln Lys
                        980 985 990
Leu Ala Phe Asn Tyr Gly Asn Ala Asn Gly Ile ser Asp Tyr Ile Asn
                995 1000 1005
Lys Trp Ile Phe Val Thr Ile Thr Asn Asp Arg Leu Gly Asp Ser
        1010 1015 1020
Lys Leu Tyr Ile Asn Gly Asn Leu Ile Asp Gln Lys Ser Ile Leu
        1025 1030 1035
Asn Leu Gly Asn Ile His Val Ser Asp Asn Ile Leu Phe Lys Ile
        1040 1045 1050
val Asn Cys Ser Tyr Thr Arg Tyr Ile Gly Ile Arg Tyr Phe Asn
        1055 1060 1065
Ile Phe Asp Lys Glu Leu Asp Glu Thr Glu Ile Gln Thr Leu Tyr
        1070 1075 1080
Ser Asn Glu Pro Asn Thr Asn Ile Leu Lys Asp Phe Trp Gly Asn
        1085 1090 1095
Tyr Leu Leu Tyr Asp Lys Glu Tyr Tyr Leu Leu Asn Val Leu Lys
        1100 1105 1110
Pro Asn Asn Phe Ile Asp Arg Arg Lys Asp Ser Thr Leu Ser Ile
        1115 1120 1125
Asn Asn Ile Arg Ser Thr Ile Leu Leu Ala Asn Arg Leu Tyr Ser
        1130 1135 1140
Gly Ile Lys Val Lys Ile Gln Arg Val Asn Asn Ser Ser Thr Asn
        1145 1150 1155
Asp Asn Leu val Arg Lys Asn Asp Gln Val Tyr Ile Asn Phe val
        1160 1165 1170
Ala Ser Lys Thr His Leu Phe Pro Leu Tyr Ala Asp Thr Ala Thr
        1175 1180 1185
Thr Asn Lys Glu Lys Thr Ile Lys Ile Ser Ser Ser Gly Asn Arg
        1190 1195 1200
Phe Asn Gln Val Val Val Met Asn Ser Val Gly Asn Asn Cys Thr
        1205 1210 1215
Met Asn Phe Lys Asn Asn Asn Gly Asn Asn Ile Gly Leu Leu Gly
        1220 1225 1230
Phe Lys Ala Asp Thr Val Val Ala Ser Thr Trp Tyr Tyr Thr His
        1235 1240 1245
Met Arg Asp His Thr Asn Ser Asn Gly Cys Phe Trp Asn Phe Ile
        1250 1255 1260
Ser Glu Glu His Gly Trp Gln Glu Lys
        1265 1270
```

1. A botulinum neurotoxin subtype E with reduced persistence having a sequence according to SEQ ID NO: 1 or SEQ ID NO: 2, or a functionally active variant thereof, for use in the treatment of a patient, wherein the patient is suffering from back pain.

2. The botulinum neurotoxin subtype E with reduced persistence for use in the treatment according to claim 1, wherein said botulinum neurotoxin subtype E with reduced persistence is a functionally active variant of a sequence according to SEQ ID NO: 1 or SEQ ID NO: 2, wherein said functionally active variant has a persistence that is at maximum 5% shorter or longer than the duration of paralysis achieved by a botulinum neurotoxin subtype E with reduced persistence with a sequence according to SEQ ID NO: 1 or SEQ ID NO: 2.

3. The botulinum neurotoxin subtype E with reduced persistence for use in the treatment of claim 1, wherein said patient is suffering from low back pain, upper back pain, neck pain or pain caused by scoliosis.

4. The botulinum neurotoxin subtype E with reduced persistence for use in the treatment of claim 3, wherein said patient is suffering from low back pain.

5. The botulinum neurotoxin subtype E with reduced persistence for use in the treatment of claim 4, wherein said patient is suffering from low back pain, wherein said patient experiences low back pain recurring within a period of from 6 weeks to 12 weeks after a first occurrence of low back pain.

6. The botulinum neurotoxin subtype E with reduced persistence for use in the treatment of claim 5, wherein said treatment comprises therapy of said first occurrence of low back pain has been selected from: (a) oral anti-inflammatory acetaminophen (APAP), NSARs; (b) mild short-acting opioids, optionally Tramadol/Ultram; (c) opioid-APAP combination products, (d) systemically acting muscle relaxing drugs; (e) aggressive physical therapy; and (f) glucocorticoid injections.

7. The botulinum neurotoxin subtype E with reduced persistence for use in the treatment of claim 1, wherein said treatment comprises administration of said botulinum neurotoxin subtype E with reduced persistence according to SEQ ID NO: 1 or SEQ ID NO: 2, or of said functionally active variant thereof, to one or more muscles selected from the group consisting of: trapezius, latissimus dorsi, rhomboideus, iliocostalis, longissimus, spinalis, semi spinalis, multifidi, rotatores, intertransversarii, erector spinae and superficial flexors (sternocleidomastoideus, anterior scalene muscles).

8. The botulinum neurotoxin subtype E with reduced persistence for use in the treatment of claim 1, wherein muscle paralysis by a botulinum neurotoxin of more than 5 weeks, optionally of more than 4 weeks, and optionally of more than 3 weeks, is contraindicated and/or deemed to be associated with negative impact on overall treatment success, optionally due to high likelihood of increased muscle atrophy.

9. A product comprising a botulinum neurotoxin subtype E with reduced persistence having a sequence according to SEQ ID NO: 1 or SEQ ID NO: 2, or a functionally active variant thereof, for use in the treatment of a patient, wherein the patient is suffering from back pain.

10. A method for treating a patient suffering from back pain comprising administering an effective amount of a botulinum neurotoxin subtype E with reduced persistence having a sequence according to SEQ ID NO: 1 or SEQ ID NO: 2, or a functionally active variant thereof.
